(19) United States
(12) Patent Application Publication
(10) Pub. No.: US 2026/0301963 A1
(43) Pub. Date: Oct. 1, 2026

(54) OBJECT EVALUATION METHOD USING COLOR ANALYSIS BASED ON BIOFLUORESCENCE IMAGES AND SYSTEM FOR THE SAME

(71) Applicant: AIOBIO CO., LTD., Seoul (KR)

(72) Inventor: Hong Cheol YOON, Seoul (KR)

(21) Appl. No.: 19/271,392

(22) Filed: Jul. 16, 2025

Related U.S. Application Data
(63) Continuation of application No. PCT/KR2025/007044, filed on May 23, 2025.

(30) Foreign Application Priority Data
May 23, 2024 (KR) 10-2024-0066797
Jul. 10, 2024 (KR) 10-2024-0091380

(51) Int. Cl. G16H 50/30; G06T 7/00; G06T 7/90; G16H 30/40; G16H 50/70

(52) U.S. Cl. CPC G16H 50/30; G06T 7/0014; G06T 7/90; G16H 30/40; G16H 50/70; G06T 2207/10064; G06T 2207/30036

(57) ABSTRACT

The present invention relates to a method of evaluating a state of a specific object, and more specifically, to a method of evaluating a specific object or target using multi-chromatic analysis based on a biofluorescence image, and a system therefor.

### CROSS-REFERENCE TO RELATED APPLICATIONS

This application is a Continuation of International Application No. PCT/KR2025/007044, filed on May 23, 2025, which claims priority under 35 U.S.C. § 119 (a) to Korean Patent Applications No. 10-2024-0066797, filed on May 23, 2024, and No. 10-2024-0091380, filed on Jul. 10, 2024. The entire disclosures of the t above-identified applications are hereby incorporated by reference herein in their entirety.

### TECHNICAL FIELD

The present invention relates to a method of evaluating a state of a specific object, and more specifically, to a method of evaluating a specific object or target using multi-chromatic analysis based on a biofluorescence image, and a system therefor.

### BACKGROUND ART

Various methods are used to evaluate the state of an object, and one of them is a method of analyzing a color confirmed from the appearance of an object. When a specific object exists, and it is possible to define a color that is seen when the object is in a good state and a color that is seen when the object is in a bad state, the states of objects of the same type can be evaluated, and the present invention has been proposed in response to demands on such a method.

For example, although oral hygiene has a significant impact on the overall body health and regular checkups and management are very important for preventing oral diseases, existing oral hygiene evaluation methods rely on subjective determination of an expert, mainly on the appearance and color of teeth that can be seen with eyes, in many cases, and thus has a problem of lacking in accuracy and objectivity. Recently, teeth images captured by applying a biofluorescence imaging technique, i. e., a technique of observing a phenomenon (fluorescence phenomenon) of absorbing light and emitting a light beam of unique light different from the initial light radiated from a light source of a predetermined wavelength by a substance produced during the metabolic process of microorganisms (e.g., microorganisms that form dental plaque) present in an object when the object is illuminated using the light source, are utilized, and therefore, it is possible to make oral hygiene evaluation and oral disease diagnosis that require considerable accuracy. However, a system of a level capable of systematically analyzing teeth images has not yet been provided and thus needs to be supplemented.

The present invention has been proposed in consideration of such problems, and relates to a method and system for quickly and accurately determining the state of an object by performing multi-color analysis on the basis of biofluorescence images.

### DISCLOSURE OF INVENTION

### Technical Problem

Therefore, the present invention has been made in view of the above problems, and it is an object of the present invention to provide a method and system for objectively evaluating s state of an object by performing color analysis based on a biofluorescence image. In particular, conventional analysis methods based on a biofluorescence image have a problem in that analysis results vary according to the researcher evaluating the object state as an automatic analysis is difficult, and they are inconvenient in actual clinical practice as slightly different values are presented each time the analysis is performed. The object of the present invention is to automate the analysis process and supplement the difficulties in evaluating parts that are darkened and brightened in color as only RGB values are used in the past, by comprehensively evaluating even Lab values.

Specifically, an object of the present invention is to objectively evaluate the state of an object, e.g., the oral hygiene state such as dental calculus, dental plaque, cavities, gum inflammation, and the like in the case of an oral cavity, by quantitatively analyzing color information of biofluorescence images.

In addition, another object of the present invention is to provide a user with accurate and detailed information on the state of an object, e.g., teeth and gums, on the basis of analysis results, and to make personalized oral health care possible.

In addition, another object of the present invention is to provide a motivation for oral health care by intuitively providing analysis results so that a user may visually check and understand the state of an object, e.g., oral health state.

In addition, another object of the present invention is to make oral health care easy in various environments such as homes, schools, and workplaces by constructing a system that can evaluate the hygiene state in an object, e.g., an oral cavity, through biofluorescence images even without specialized knowledge.

Meanwhile, the technical problems of the present invention are not limited to the technical problems mentioned above, and unmentioned other technical problems can be clearly understood by those skilled in the art from the following descriptions.

### Technical Solution

To accomplish the above objects, according to one aspect of the present invention, there is provided a method of evaluating an object using color analysis based on a biofluorescence image by a computing device including a central processing unit and a memory, the method comprising the steps of: receiving the biofluorescence image of an arbitrary object; acquiring RGB values and Lab values of an evaluation area in the biofluorescence image; and performing color analysis on the basis of the acquired RGB values and Lab values, and displaying a result of the color analysis performed on the object on a specific coordinate system.

In addition, the method of evaluating an object using color analysis may further comprise, after the step of receiving the biofluorescence image, the step of identifying only the evaluation area from the biofluorescence image.

In addition, in the method of evaluating an object using color analysis, the color analysis may include a process of determining a color cluster to which a plurality of points within the evaluation area belongs by comparing the acquired color information with a previously generated color cluster.

In addition, in the method of evaluating an object using color analysis, the x-axis of the coordinate system may represent a ratio of a G value to an R value, and the y-axis may represent an L value.

In addition, in the method of evaluating an object using color analysis, the coordinate system may include a plurality of color clusters defined in advance.

In addition, in the method of evaluating an object using color analysis, the step of displaying a result of the color analysis may include a step of displaying points mapped to a plurality of points within the evaluation area on the coordinate system.

In addition, in the method of evaluating an object using color analysis, the step of displaying a result of the color analysis further may include a step of determining to which color clusters the plurality of mapped points belong, and calculating a ratio of the color clusters to which the plurality of points belong.

In addition, in the method of evaluating an object using color analysis, the step of displaying a result of the color analysis may further include a step of determining to which risk group among a high-risk group, a medium-risk group, and a low-risk group the object belongs according to the ratio of the color clusters to which the plurality of points belong.

According to another aspect of the present invention, there is provided a method of clustering for color analysis based on a biofluorescence image by a computing device including a central processing unit and a memory, the method comprising the steps of: collecting a plurality of biofluorescence images; identifying only an evaluation area to be analyzed in each of the biofluorescence images; acquiring RGB values and Lab values of the evaluation area in the biofluorescence images; displaying all color distributions corresponding to the evaluation area on a coordinate system on the basis of the acquired RGB values and Lab values; determining the number of clusters and a representative value of each cluster; and displaying RGB values corresponding to the representative value of each cluster on the coordinate system.

According to another aspect of the present invention, there is provided a system including a central processing unit and a memory, wherein the central processing unit executes instructions for performing an object evaluation method stored in the memory, and the object evaluation method may include the steps of: receiving the biofluorescence image of an arbitrary object; acquiring RGB values and Lab values of an evaluation area in the biofluorescence image; and performing color analysis on the basis of the acquired RGB values and Lab values, and displaying a result of the color analysis performed on the object on a specific coordinate system.

### Advantageous Effects

According to the present invention, there is an effect of objectively and quantitatively evaluating an object through color analysis based on a biofluorescence image.

In particular, there is an effect of accurately identifying presence and severity of various oral diseases such as dental calculus, dental plaque, cavities, and gum inflammation, getting out of existing subjective evaluation methods in evaluating the oral hygiene state.

In addition, the present invention allows a user to intuitively understand oral health states of his or her own by providing analysis results in a user-friendly way, and ultimately has an effect of contributing to prevention of oral diseases by increasing awareness of a user about the oral health states and inducing active participation in oral health care.

Meanwhile, the effects of the present invention are not limited to those mentioned above, and unmentioned other technical effects will be clearly understood by those skilled in the art from the following descriptions.

### BRIEF DESCRIPTION OF THE DRAWINGS

FIG. 1 is a conceptual view showing the overall concept of an object evaluation method and system according to the present invention.

FIG. 2 is a flowchart illustrating an object evaluation method according to an embodiment of the present invention.

FIG. 3 is a view showing a coordinate system utilized in an object evaluation method according to the present invention.

FIG. 4 is a view showing the oral hygiene state of a certain patient through a coordinate system, and shows the state of a patient whose oral hygiene state belongs to a high-risk group.

FIG. 5 is a view showing the state of a patient whose oral hygiene state belongs to a medium-risk group.

FIG. 6 is a view showing the state of a patient whose oral hygiene state belongs to a low-risk group.

FIG. 7 is a flowchart illustrating a pre-clustering method for color analysis according to another embodiment of the present invention.

FIG. 8 is a view showing a medical support system according to another embodiment of the present invention.

FIG. 9 is a view showing a medical support method according to another embodiment of the present invention.

FIGS. 10 to 13 are views showing an example of interfaces that a medical support system provides to a user (diagnostician) according to the present invention.

### BEST MODE FOR CARRYING OUT THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

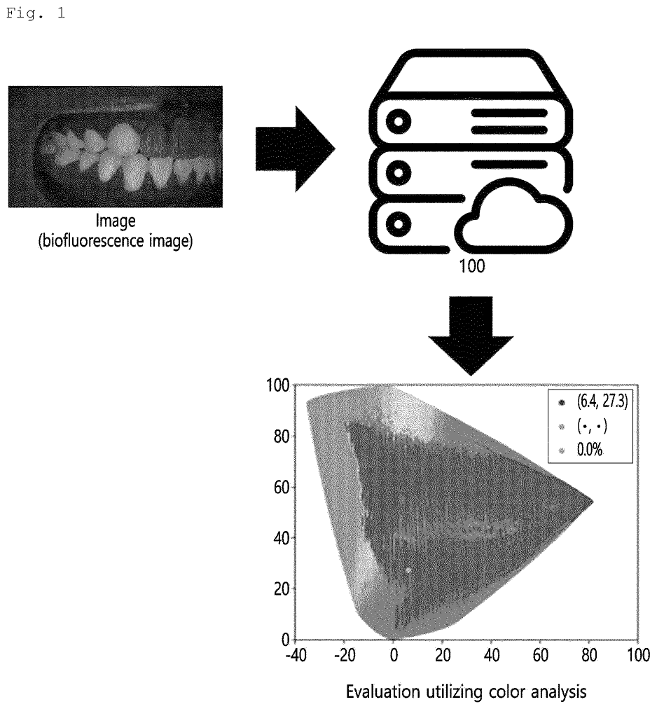

FIG. 1 is a view for understanding the overall concept of an object evaluation method and system according to an embodiment of the present invention. The system may basically include a computing device 100, and when a biofluorescence image of a specific object is input, the computing device 100 may perform multi-chromatic analysis and output an evaluation result on the state of the object as shown in the drawing.

For example, when an image of the teeth of a patient visiting a dental clinic is captured using a biofluorescence imaging device and input into the computing device 100, the method according to the present invention may be utilized to evaluate the presence and severity of dental calculus, dental plaque, and cavities by segmenting the teeth and gum areas and analyzing color information of a teeth area to be evaluated. The computing device 100 provides results of the evaluation to a dentist, and the dentist may establish a customized diagnosis and treatment plan for the patient on the basis of the results. For example, when the dental calculus is severe, scaling may be recommended, or when cavities are suspected, additional examinations may be performed.

As another example, the present invention may be implemented to allow a general consumer or a food inspector to input a food image captured using a biofluorescence imaging device into the computing device 100 to check freshness of the food and see results of evaluation thereon. The method according to the present invention evaluates freshness, a degree of spoilage, or the like by analyzing color information of the food, and provides a result thereof to the consumer or inspector. The consumer or inspector may make a further wiser choice when purchasing food by referring to the evaluation result. For example, when freshness of fruit is evaluated as low, the evaluation result may be used to withhold purchase and sale, or when the degree of spoilage of meat is evaluated as high, the evaluation result may be used to decide whether or not to discard the meat.

As described above, the basic configuration of the present invention is implemented to provide a user with a color analysis result when there is a biofluorescence image of an object to be evaluated, and hereinafter, the process of evaluating an object by a system including the computing device 100 will be described in more detail.

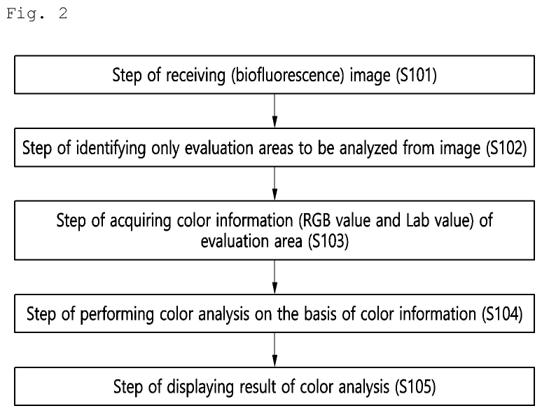

FIG. 2 is a flowchart illustrating a method of evaluating an object using color analysis according to an embodiment of the present invention. An embodiment of the present invention will be described in detail with reference to FIG. 2.

Before describing in detail, it should be understood that the object evaluation method according to the present invention may be executed by the computing device 100 having a central processing unit and a memory. The type of the computing device may include both portable terminals such as a smart phone, a PDA, and a tablet PC, and terminals fixedly placed in a predetermined location such as a desktop PC. The central processing unit may also be referred to as a controller, a microcontroller, a microprocessor, a microcomputer, or the like. In addition, the central processing unit may be implemented as hardware, firmware, software, or a combination thereof. In the case of implementing the method using hardware, the central processing unit may be implemented as an application specific integrated circuit (ASIC), a digital signal processor (DSP), a digital signal processing device (DSPD), a programmable logic device (PLD), a field programmable gate array (FPGA), or the like, and in the case of implementing the method using firmware or software, the firmware or software may be configured to include modules, procedures, functions, and the like that perform the functions or operations described above. In addition, the memory may be implemented as Read Only Memory (ROM), Random Access Memory (RAM), Erasable Programmable Read Only Memory (EPROM), Electrically Erasable Programmable Read-Only Memory (EEPROM), flash memory, Static RAM (SRAM), Hard Disk Drive (HDD), Solid State Drive (SSD), or the like.

In some cases, the computing device 100 may be a server, and in this case, the server may be a device that stores and executes a program, i.e., a set of instructions, for actually implementing the object evaluation method according to the present invention. The server may be at least in the form of one server PC managed by a specific user, or may be in the form of a cloud Server provided by another company, i.e., a cloud server that a user may use after registering as a member.

In addition, in some cases, the method according to the present invention may be executed on a cluster system configured of a plurality of computing devices rather than the single computing device 100. Within the cluster system, a plurality of computing devices that need to execute the object evaluation method may be set to perform different operations, respectively.

Hereinafter, various related embodiments will be described with reference to the drawings.

Referring to the drawings, the object evaluation method according to the present invention starts from a step of receiving a biofluorescence image from a user or an external device by the computing device 100 (S101). The biofluorescence image refers to an image capturing a fluorescence phenomenon generated by microorganisms (bacteria) or the like existing on an object when light of a specific wavelength selected from the range of visible light is radiated toward the object. For example, a porphyrin substance generated during the metabolic process of microorganisms may exhibit a fluorescence phenomenon in blue light of 380 to 420 nm, and an image capturing this phenomenon may be defined as a biofluorescence image. The process of capturing the biofluorescence image may further include a process of passing the image through a color filter, but since this is not a matter to be discussed in detail in the detailed description, it will be described briefly herein.

Meanwhile, the image received at this step is not limited to biofluorescence images capturing the teeth. For example, biofluorescence images captured to evaluate freshness of food may be utilized. Biofluorescence images acquired targeting food may be used to detect moisture content and changes in the texture of the food, and provide freshness information that is difficult to check with naked eyes, and the biofluorescence images of food may also be received at this step. In addition, biofluorescence images captured to analyze skin conditions may also be received.

Meanwhile, the computing device 100 may receive the biofluorescence images in various ways. For example, a user may transmit a biofluorescence image captured using a mobile device such as a smartphone, a tablet PC, or the like to the computing device through a wireless communication method such as Bluetooth, Wi-Fi, or the like. Alternatively, the user may connect a dedicated biofluorescence photographing device to the computing device 100 by wire and directly transmit the image.

After step S101, the computing device 100 may perform an operation of identifying only an area to be evaluated, i.e., evaluation area, from the image (S102). When the object to be evaluated is teeth, it is highly probable that the biofluorescence image is an image that captures the inside of an oral cavity, and in this case, the computing device 100 may perform an operation of identifying only the surface of the teeth as an evaluation area to accurately evaluate the object.

At this step, various types of algorithms may be utilized. For example, since the color difference between the teeth and gums is comparatively distinct, a color-based segmentation algorithm that separates the two areas using color information can be utilized. That is, a task of distinguishing an evaluation area may be performed by utilizing an algorithm that analyzes color distribution of the teeth and gums in various color spaces such as RGB, HSV, Lab, and the like, and distinguishes the two areas using a threshold setting or clustering technique. In addition, a morphological operation algorithm that emphasizes the shape of the teeth and clearly distinguishes the boundary with the gums by applying a morphological operation may also be utilized. For example, identification of the evaluation area can be accomplished by removing fine curves on the teeth surface through an erosion operation and expanding the teeth area through an expansion operation to clarify the boundary with the gums. As another example, it may be implemented to find the boundary between the teeth and gums using an edge detection algorithm. The edge detection algorithm borrows a method of finding a part where the change of brightness is large in the image, and since the boundary between the teeth and the gums has a big difference in the brightness, the bright part can be effectively separated through the edge detection. In addition, an active contour algorithm may be utilized. This algorithm is characterized by setting an initial contour line and extracting the boundary of an object by transforming the outline on the basis of the features of the image. A rough shape of the teeth is set as the initial contour line, and the evaluation area can be identified by transforming the contour line in accordance with the boundary of the teeth using information such as the color, brightness, and the like of the biofluorescence image.

Meanwhile, it is most preferable to segment the teeth area in the biofluorescence image using a Deep Learning-based Segmentation algorithm, more specifically, a deep learning algorithm such as U-Net, Mask R-CNN, or the like. The deep learning algorithm may learn the features of the teeth and gums through a large amount of learning data, and accurately segment the teeth area in a new image on the basis of the learned features. Although it will be described below, as the present invention may be implemented to utilize the results of analyzing a large amount of biofluorescence images stored in a database in advance for generation of a coordinate system and deep learning, it is most preferable to use a learning-type algorithm like this in identifying an evaluation area.

Meanwhile, the computing device may accurately identify an evaluation area from the biofluorescence image by using the various algorithms mentioned above alone or in combination. For example, after roughly separating the teeth and gums first through color-based segmentation, the boundary can be extracted more accurately by applying an edge detection or active contour model. In addition, the initial segmentation result can be improved by using a segmentation model based on deep learning, or a segmentation result can be post-processed by applying a morphological operation.

After step S102, the computing device 100 may execute a step of acquiring color information of an evaluation area in the biofluorescence image (S103). The color information may include an RGB value and a Lab value. The RGB value is a value that expresses a color in combination of three color components of red (R), green (G), and blue (B), and the Lab value is a value that expresses a color by three values of brightness (L), green-red axis (a), and blue-yellow axis (b). At this point, the RGB value includes all values that can be detected from an image in which teeth are identified before. In addition, for reference, the Lab value has an advantage of expressing a color difference more accurately as it is designed to be similar to color perception of human being.

Meanwhile, after step S103, a step of performing color analysis, i.e., multi-color analysis, on the basis of the color information (S104) may be continued. This step may be more precisely understood as a process of mapping color information obtained from an evaluation area to a unique coordinate system.

Specifically, at step S104, the computing device 100 may perform an operation of mapping all color information that can be extracted from an evaluation area onto a predefined coordinate system, in which the x-axis represents the ‘a’ value (the degree of green-red) among the Lab value and the y-axis represents the L value (brightness) among the Lab value.

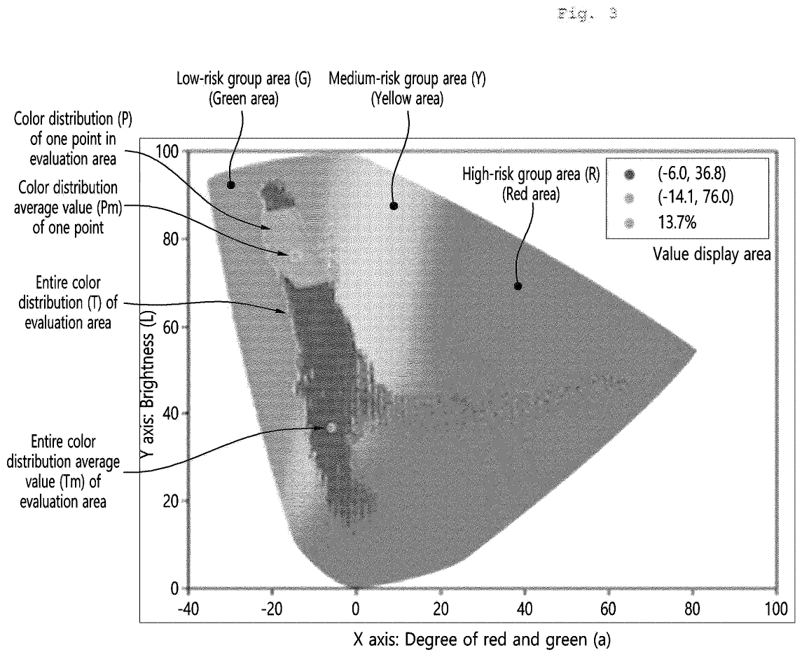

FIG. 3 shows an example of a coordinate system utilized in the present invention. FIG. 3 is a view showing a result of analyzing an evaluation area (teeth surface) of a specific object (inside the oral cavity of a patient) on the unique coordinate system proposed in the present invention.

Referring to FIG. 3, the coordinate system is a two-dimensional coordinate system, in which the x-axis represents the ‘a’ value (the degree of green-red) among the Lab value and the y-axis represents the L value (brightness) among the Lab value as mentioned above, and it can be confirmed that the entire color distribution (T) of the evaluation area is displayed on the coordinate system in units of pixels on the basis of the color information acquired from the evaluation area. That is, the T area indicated on the drawing may also be defined as a set of blue dots, and each blue dot is mapped by a pair of L value and ‘a’ value obtained on the basis of the color information obtained from the evaluation area (teeth surface). The items displayed on this coordinate system, especially the distribution map of a specific shape, include all values that can be displayed by the biofluorescence from a target (e. g., the oral cavity of a patient). From these values, the range of L value and distribution of green and red can be confirmed, and in particular, the range of L value may be utilized as an important indicator for evaluating wear or cracks of teeth in the oral cavity, and further, may be utilized as an important indicator for evaluating enamel and dentin. As a matter to be noted, in the multi-color analysis according to the present invention, the B value among the RGB value and the b value among the Lab value are not used, and it may be implemented to set an offset value for the B value or b value as needed. Exclusion of the B value or b value has an effect of reducing information not necessarily needed for color analysis, and accordingly has an effect of reducing loads of operation of the computing device 100. In addition, exclusion of the b value among the Lab value has a technical meaning in that the evaluation is performed only on the fluorescent area generated by blue light.

Meanwhile, as a point (Tm) indicating the average value of the entire color distribution in a corresponding area is displayed in the entire color distribution (T) area of the evaluation area, the user may intuitively determine the state of the evaluation area. In addition, a subarea corresponding to a normal area may be separately searched from the evaluation area, and this may be searched according to conditions set in advance by the user or searched by a learned artificial intelligence algorithm. For the normal area, the average value of the area may be defined as Ga, Ra, and La.

In addition, when a point in the evaluation area is specified, color distribution P of the point may be displayed as a partial area as shown in the drawing, and similarly, the average value Pm of the partial area of the point may also be displayed.

In addition, a separate value display area, in which specific analysis values of the evaluation area are displayed, may exist at the upper right of the coordinate system. In the drawing, it can be confirmed that Tm, Pm, and the ratio of the color distribution of one point to the color distribution area of the entire evaluation area are displayed. Other calculated values may also be displayed in the value display area. As these values are acquired, various quantitative evaluations can be made. For example, when the difference between the maximum value and the average value in a predetermined area is placed in the denominator, and the difference between the maximum value and the value of examination at one point is placed in the numerator, the state of teeth may be quantitatively evaluated.

Meanwhile, as a matter to be noted, it can be seen that the coordinate system of the present invention defines arbitrary risk group areas configured of a low-risk group area G, a medium-risk group area Y, and a high-risk group area R as shown in the drawing. These risk group areas are defined as a result of clustering in advance based on a large number of biofluorescence images, and as will be described again in the description below, the clustering process may be performed by plotting the L value and ‘a’ value among the Lab values acquired from a large number of biofluorescence images on a coordinate system to indicate all areas that the teeth may have, determining a representative value for each cluster on the basis of a predetermined number of clusters, and displaying the R value and G value corresponding to each representative value on the coordinate system. A matter to be noted is that RGB values are used in the pre-clustering process, and among the values, only the R value and G value are used, and use of the B value is excluded herein as well.

In addition, for reference, although the same object is photographed, the values displayed in the coordinate system may be displayed as different values according to the photographing device (camera) used to photograph the target (object). However, it is understood that the detailed description is made without considering such a situation on the premise that the object is photographed using the same photographing device (camera). However, when a target (object) is photographed using different photographing devices, a process of normalizing the result values by scaling the acquired values (RGB, Lab) within a reference range, if necessary, may be included.

A coordinate system utilized in the present invention has been described above with reference to FIG. 3.

Returning to the description of FIG. 2, after the multi-color analysis is performed on the coordinate system at step S104, a step of displaying a color analysis result to the user (S105) may be executed. This step may be understood as a step of outputting the color analysis result displayed on the coordinate system through a display device (not shown) by the computing device 100, or may also be understood as a step of displaying the color analysis result through a display means of another terminal connected to the network by the computing device 100.

An object evaluation method according to an embodiment of the present invention has been described above with reference to FIGS. 2 and 3.

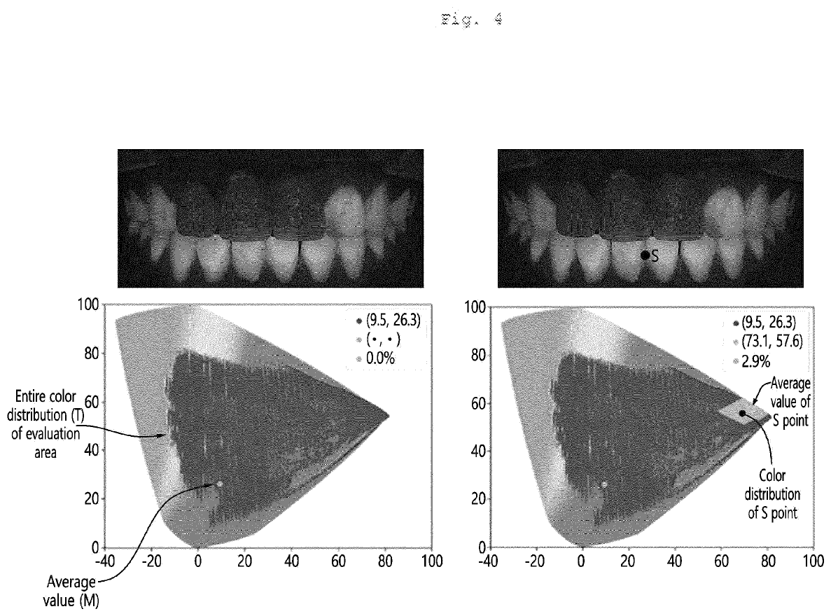

FIGS. 4 to 6 show the results of multi-color analysis case by case, and it is intended to explain how the color analysis result marked on the unique coordinate system mentioned above can be actually utilized.

First, FIG. 4 is a view showing a case of evaluating the oral hygiene state of a patient belonging to the high-risk group area R. FIG. 4 (a) is a view showing a color analysis result when a biofluorescence image obtained by photographing the oral cavity of a patient (front side of teeth) is received. Referring to the color analysis result displayed in the coordinate system, it can be seen that the evaluation area of the patient, i.e., the teeth surface, is the reddish area showing a view of skewed color distribution, and this can be easily inferred from the fact that the red fluorescent part, i.e., the area where microorganisms exist, appears to be considerably wide in the biofluorescence image of the patient. Seeing in the coordinate system, although color distribution (T) of the evaluation area (teeth surface) is distributed across the low-risk group area G, the medium-risk group area Y, and the high-risk group area R, when it is examined in which group area the evaluation area is most distributed, it can be seen that it is overwhelmingly distributed in the high-risk group area R, and through this, the user (e. g., dentist) may intuitively know that the patient is in a high-risk group of a considerably poor oral hygiene state.

FIG. 4 (b) is a view additionally showing the color distribution of one point (point S) on the coordinate system when the one point is specified in the biofluorescence image. Referring to the drawing, it can be confirmed that a color distribution area corresponding to point S is displayed (light purple area) on the coordinate system and an average value of the point is also displayed. FIG. 4 (b) may be a view output on the coordinate system when, for example, the user (e. g., dentist) clicks on a point(S) on the biofluorescence image using an input device such as a mouse or the like. In this way, the user may click specific points on the biofluorescence image and show the patient the color distribution each time the point is clicked to help the patient understand the oral hygiene state.

Meanwhile, as a matter to be noted in FIG. 4, the patient has a dental prosthesis (implant), and it may be implemented to display the color distribution corresponding to the dental prosthesis in the black area at the bottom of the coordinate system. In the biofluorescence image, a dental prosthesis, which is not a natural tooth, is displayed dark, and by utilizing this characteristic, it is possible to grasp how much area the dental prostheses occupy in the evaluation area on the coordinate system, i. e., how many dental prostheses are placed, through only the coordinate system.

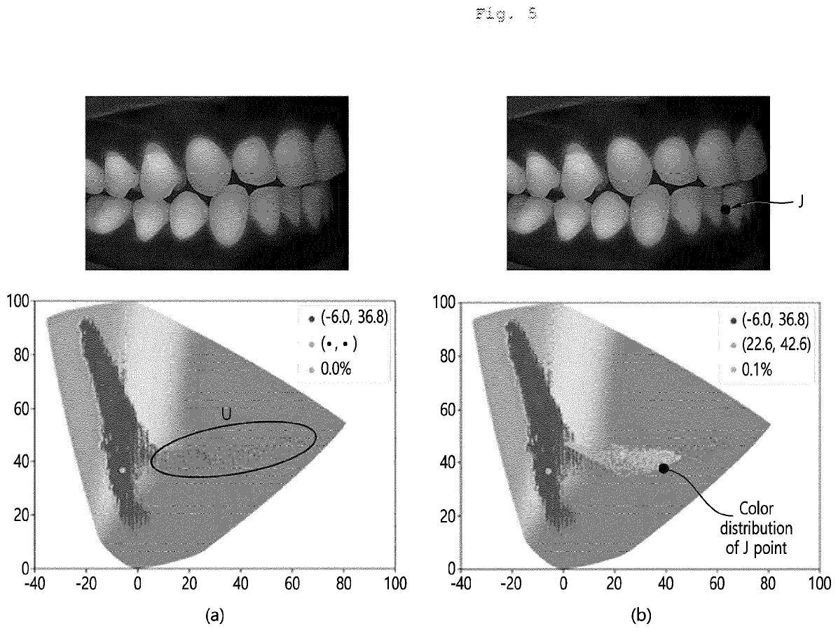

FIG. 5 is a view showing the oral hygiene state of a patient when a considerable part of the color distribution belongs to the medium-risk group area Y. Examining the biofluorescence image of the patient, most of the patient's teeth area (evaluation area) is cleanly and well managed. However, it can be seen that there is a small plaque (red fluorescence) area at the lower right corner. Looking at the coordinate system shown in FIG. 5 (a), it can be seen that although most of the color distribution of the patient falls in the low-risk group area G and the medium-risk group area Y, a set of some blue dots is distributed across the high-risk group area R. That is, due to the presence of the small plaque area mentioned above, the U area may be clearly displayed on the coordinate system in the color analysis result, and through this, the user may intuitively grasp the state of the patient.

FIG. 5 (b) is a view showing the appearance of the plaque area by specifying a point (point J), and it can be confirmed in the coordinate system of (b) that color distribution of the area corresponding to point J is displayed in a different color (light purple).

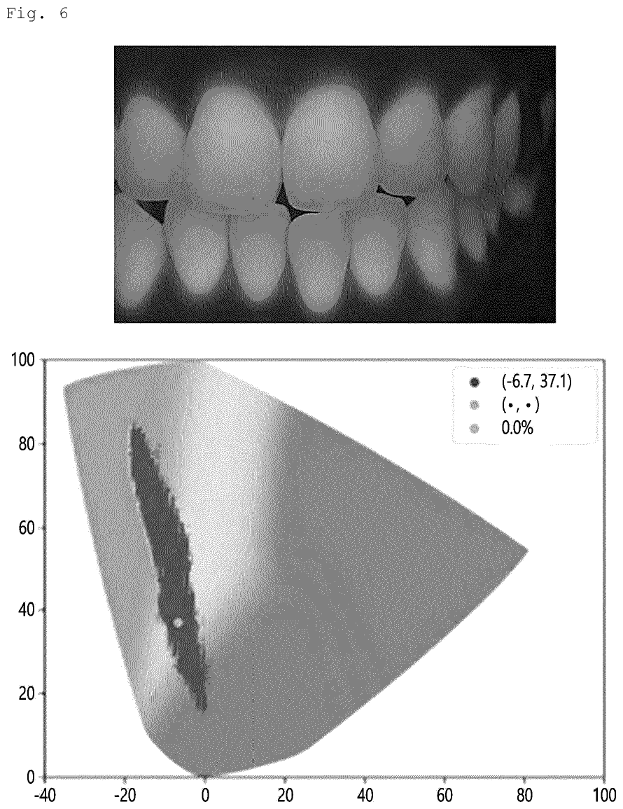

FIG. 6 is a view showing evaluation of an oral hygiene state of a patient belonging to the low-risk group area G. As can be seen in the biofluorescence image, the patient does not show red fluorescence at all, and looking at the color analysis result, it can be seen that in all areas, color distribution is shown only in the low-risk group area G and the medium-risk group area Y, and color distribution is not shown at all in the high-risk group area R.

Examples of utilizing the result of multi-color analysis displayed on the coordinate system are described above with reference to FIGS. 4 to 6.

Meanwhile, it can be implemented to define a color distribution area for each disease by learning, through an artificial intelligence algorithm, which disease matches the color analysis result according to the multi-color analysis. That is, as quantitative evaluation can be performed on the examination areas, and supervised or unsupervised learning of the artificial intelligence algorithm is possible by utilizing the quantitative evaluation, each time an image of a target is captured or each time a state of the target is diagnosed based on the captured image, it can be learned that which disease matches the color analysis result.

Specifically, when the result of analysis obtained at step S104 is provided to a user (e.g., dentist), and the user determines a disease name or a diagnosis name as a result of actually observing the inside of the oral cavity of the patient with naked eyes, it is possible to map which disease name or diagnosis name corresponds to the previously analyzed result, i.e., the color distribution, and it can be implemented to accomplish the learning through the mapping by an artificial intelligence algorithm. In this way, when a specific color distribution area is obtained as a result of examination, it is possible to grasp which disease the patient is likely to have or which diagnosis he or she is likely to receive, and as the detailed information grasped in this way is directly delivered to the user (e. g., dentist) or the patient, an effect of improving the accuracy and speed of the overall treatment can be obtained. In addition, when the color distribution is mapped to a specific disease name or diagnosis name by learning of the artificial intelligence algorithm, the user or patient may select a specific one point or a specific area on the coordinate system, and receive a result of evaluation on the area, and as chatbot chatting is allowed according to implementation, the user or patient may accurately and easily know information about the state.

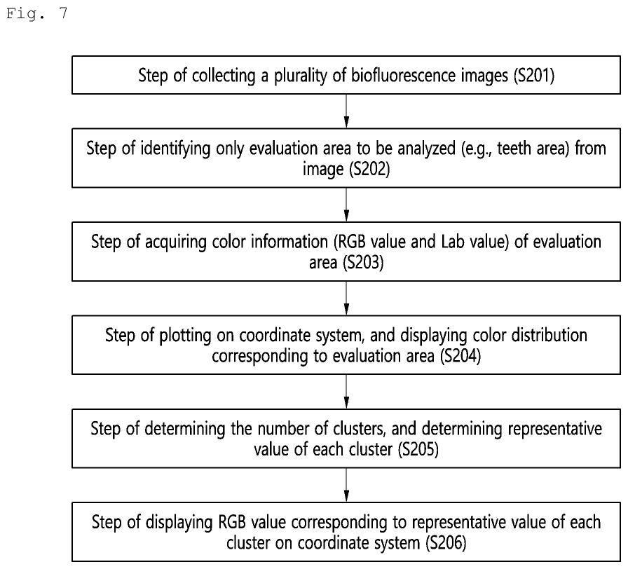

FIG. 7 relates to another embodiment of the present invention, and sequentially shows a pre-clustering method for color analysis. An embodiment of the present invention will be described in detail with reference to FIG. 7.

The clustering method first includes a step of collecting a plurality of biofluorescence images (S201). To help understanding of the invention, it will be described, in the detailed description, on the basis of an example of utilizing an image captured using a dental biofluorescence imaging device to evaluate the state of teeth in the oral cavity. For example, the computing device 100 may collect images captured using various types of biofluorescence imaging devices such as a device for examining dental caries activity, a device for examining dental plaque activity, a device for detecting dental calculus, and the like.

After step S201, the computing device 100 may identify only an evaluation area to be analyzed in each of the collected biofluorescence images (S202). The evaluation area means a specific area in the biofluorescence image for evaluating the oral health state through color analysis. For example, in the case of a teeth biofluorescence image, the teeth surface may be set as the evaluation area. The evaluation area may be set by the user himself or herself or automatically extracted through an image processing technique. For reference, this step does not necessarily need to be performed by the computing device 100 and may also be performed by utilizing an external service server.

After step S202, the computing device 100 may acquire RGB values and Lab values of the evaluation area in the biofluorescence images (S203). The RGB value represents the values of the red (R), green (G), and blue (B) components of each pixel, and the Lab value represents the values of brightness (L), green-red axis (a), and blue-yellow axis (b) of each pixel. The RGB values are the most basic way of expressing the color of an image, and the Lab value has an advantage of expressing a color difference more accurately as it is designed to be similar to color perception of human being. As mentioned above briefly, the present invention excludes use of the B value among the RGB value, and excludes use of the b value among the Lab value. In addition, although an offset value may be set for the B value or b value on the coordinate system to help visual understanding of the user, except these cases, the B value or b value does not contribute to an actual analysis process.

After step S203, the computing device 100 may display all color distributions corresponding to the evaluation area on the coordinate system on the basis of the acquired RGB values and Lab values (S204). Precisely, at this step, the L and ‘a’ values among the Lab value of each pixel are expressed on a two-dimensional coordinate system, and all of these points are displayed to visualize the color distribution.

After step S204, the computing device 100 may determine the number of clusters and a representative value of each cluster (S205). Clustering is a process of grouping pixels having similar colors, and although clustering according to the present invention utilizes the K-means clustering algorithm, it is understood that this algorithm does not necessarily need to be utilized. The number of clusters can be determined by any algorithm, and preferably, it may be determined to uniformly arrange the clusters on the coordinate system on the basis of the overall color distribution state. In addition, the representative value of each cluster may be determined as the coordinate value of the cluster center point.

After step S205, the computing device 100 displays RGB values corresponding to the representative value of each cluster on the coordinate system (S206). At this step, the representative value of each cluster is converted into an RGB color space and displayed on the coordinate system. Through this, it may be visually confirmed which color range is represented by each cluster.

Hereinafter, a medical support system and a method thereof according to another embodiment of the present invention will be described.

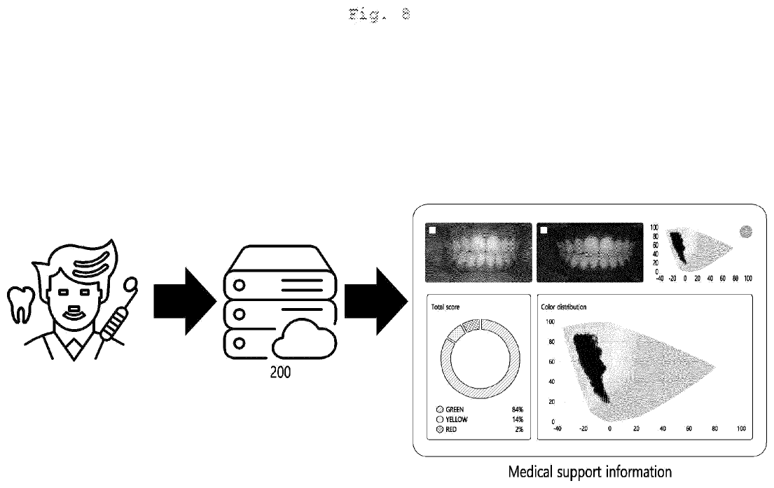

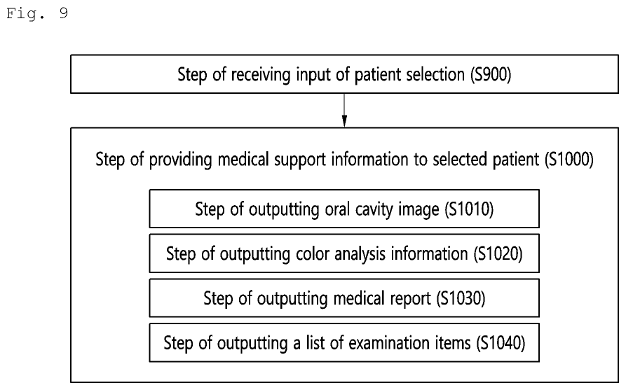

FIGS. 8 and 9 are views for understanding the overall concept of a medical support service and a medical support method according to another embodiment of the present invention. Although the service may be executed by the computing device 100 described above in FIG. 1, it is not necessarily limited thereto. However, it is assumed in the detailed description that the computing device 100 provides even the medical support service described below to help understanding.

Referring to FIG. 8, when a biofluorescence image photographing the oral cavity of a specific patient is input, the computing device 100 may perform multi-chromatic analysis, and provide a result of evaluation on the state of the patient in the form of medical support information as shown in the drawing.

As described above, the basic configuration of this embodiment is to provide a user (diagnostician) with a result of color analysis when there is a biofluorescence image of a patient to be evaluated, and hereinafter, it will be discussed in more detail the interfaces provided by the system that includes the computing device 100 and supports the diagnosis process of a user (diagnostician).

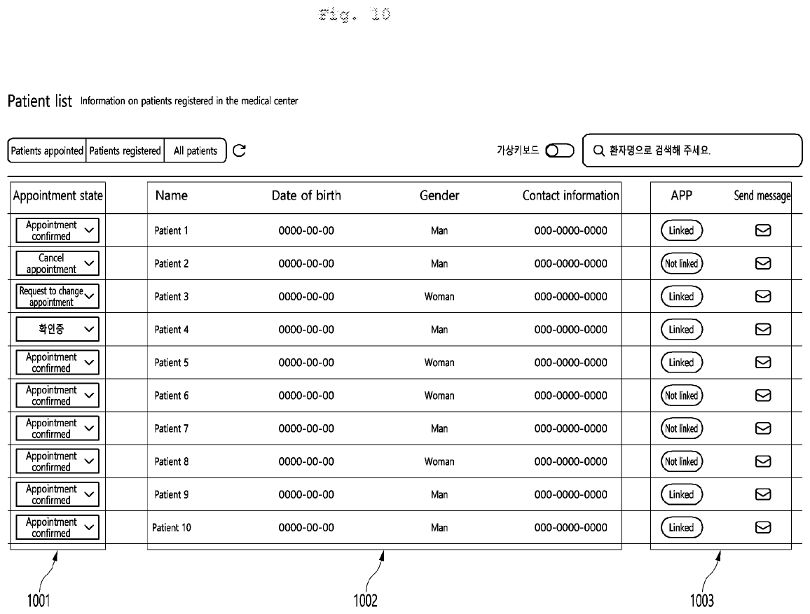

Referring to FIG. 9, the medical support method includes a step of receiving a patient selection input by the computing device 100 (S900). This step may be understood as a step of receiving an input by a user through the interface shown in FIG. 10. Referring to FIG. 10, a patient list may be output on the monitor screen that the user sees, and this screen may display an area (1001) that shows the appointment state of each patient, an area (1002) that shows information such as the name, date of birth, gender, and contact information, and additionally, an area (1003) that shows an indication of whether the patient has downloaded and linked a specific APP (e.g., an APP only for patients to be provided with treatment-related information in association with a diagnosis support system, etc.), a button for opening a function of contacting each patient by means of text message, and the like.

Referring to FIG. 9 again, after step S900, a step of providing medical support information to a selected patient (S1000) may be executed. This step includes detailed steps of analyzing an oral cavity image of a patient and outputting color analysis information, a medical report, a list of examination items, and the like by the computing device 100, and the information provided to the user (diagnostician) at this step will be collectively referred to as medical support information. That is, the medical support information includes all types of information that the user (diagnostician) may refer to when treating a patient, and in particular, the medical support information includes color analysis information, a medical report, a list of examination items, and the like among these.

Step S1000 first includes a step of outputting an oral cavity image (S1010), and this step may be performed on the premise that a biofluorescence image is acquired by a user (diagnostician) by capturing the oral cavity of the patient.

Meanwhile, the image output at this step is not limited to the biofluorescence images capturing the teeth, and may also include images capturing the inside of the oral cavity of the patient in a usual way.

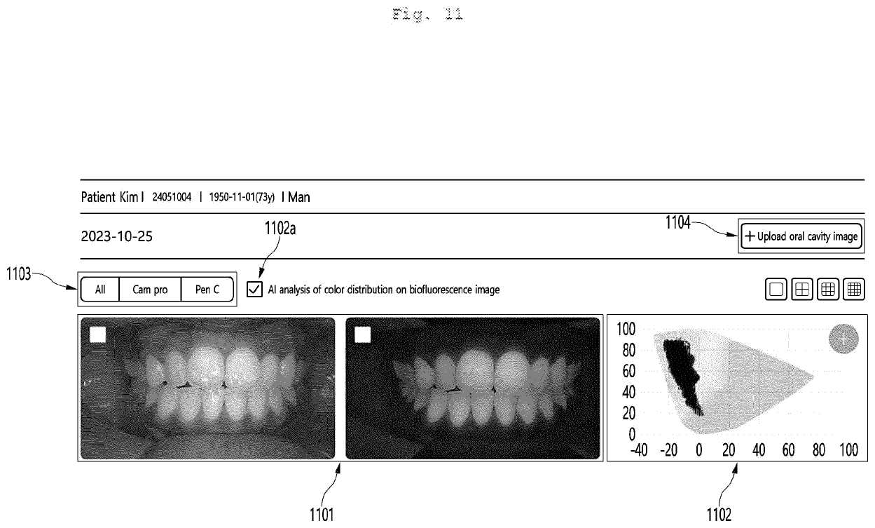

FIG. 11 is a view showing a screen that outputs an oral cavity image of a patient to a user (diagnostician). Referring to the drawing, it can be confirmed that images capturing the oral cavity of the patient in a usual way and oral cavity images (1101) of the patient such as biofluorescence images are displayed on the screen. In addition, as a color analysis result that will be described below, particularly, a color analysis result image (1102) that may easily confirm the result analyzed by artificial intelligence may be displayed together.

In addition, referring to FIG. 11, a selection area 1103 (a selection button for selecting an image captured using a photographing device (cam pro, Pen C, etc.)) capable of selecting a type of oral cavity image may be provided for convenience of the user (diagnostician), and a button (1102a) for selecting whether or not to display together the color analysis result of the fluorescence image, a button (1104) for additionally uploading an oral cavity image, and the like may be provided.

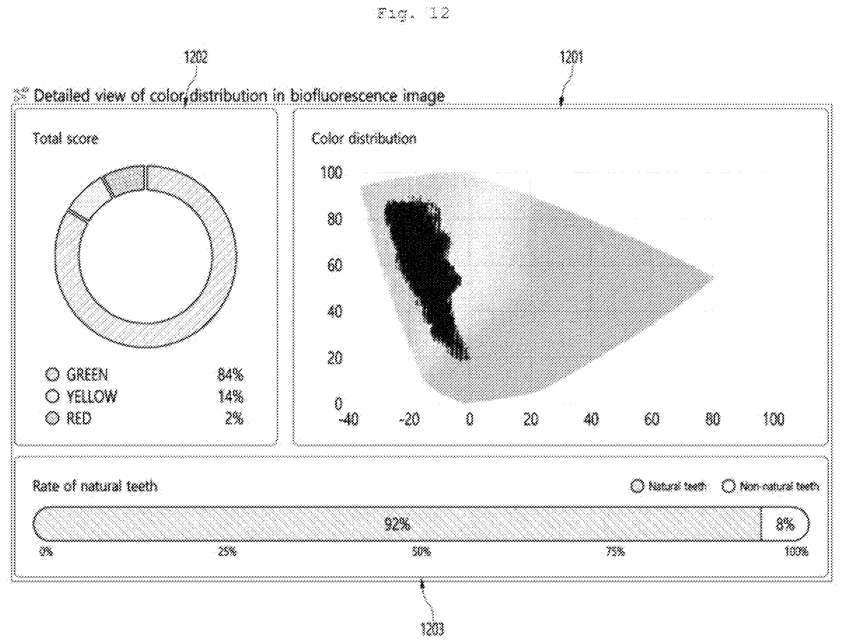

After step S1010, a step of outputting color analysis information (S1020) is executed. FIG. 12 shows an example of color analysis information provided to a user (diagnostician). Referring to the drawing, it can be confirmed that the color analysis information includes information such as a color distribution map 1201, a total score 1202, a natural teeth ratio 1203, and the like. Among these, the color distribution map 1201 may be considered as core information among the color analysis information, and the total score, the natural teeth ratio, and the like may be information provided for the user (diagnostician) to more intuitively understand the state of a patient.

Among these, the color distribution map 1201 may be obtained as a result of an analysis operation of the computing device 100, and the analysis operation of the computing device 100 may include a step of identifying only an evaluation area to be analyzed from the biofluorescence image, a step of acquiring color information (RGB values and Lab values) of the evaluation area, and a step of performing color analysis based on the color information. For these steps, refer to the contents described above through FIGS. 3 to 6.

Meanwhile, referring to FIG. 12, the total score 1202 of the oral hygiene state of a patient may be calculated based on previously analyzed results, and in particular, since the ratios of high-risk group, low-risk group, and medium-risk group may be identified through multi-color analysis for generating the color distribution map 1201, the total score may be calculated on the basis of the ratios. In addition, the natural teeth ratio 1203 may be obtained by calculating a result obtained through the multi-color analysis, i.e., the ratio of natural teeth to non-natural teeth (dental prosthetics).

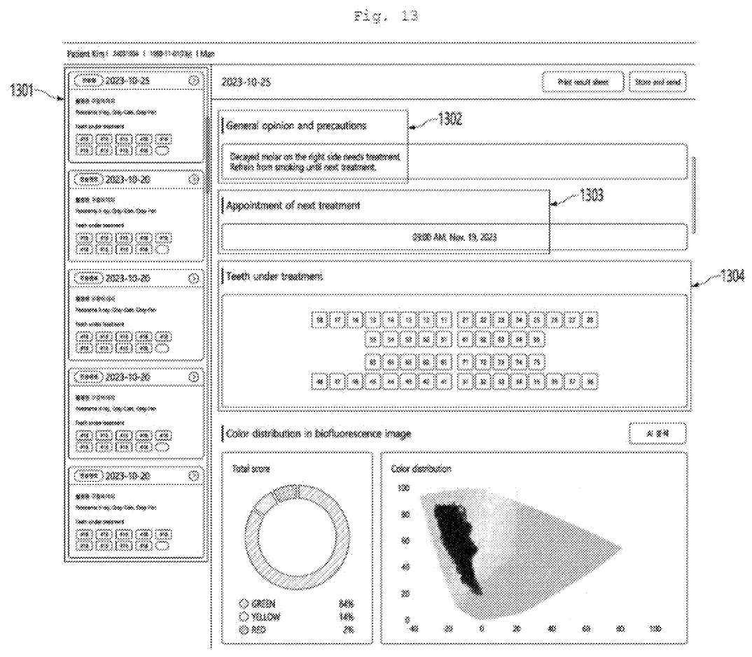

Referring to FIG. 9 again, the step of providing medical support information (S1000) includes a step of outputting a medical report (S1030), and this will be described with reference to FIG. 13.

Referring to the drawing, the medical report may include daily checkup history of a patient (1301). This may include information on the type of images photographing the oral cavity of the patient (or the type of a device capturing the image), and information on the teeth examined.

In addition, the medical report may include an opinion (1302) generated based on determination of the computing device 100 of its own (determination through an artificial intelligence algorithm), and this may include at least information on the teeth that need care, and may additionally include measures to be taken for dental health.

In addition, the medical report may further include next treatment schedule of the patient 1303, a teeth selection area 1304 for inputting teeth currently under treatment, and the like, and through this, convenience of the user (diagnostician) in the treatment process can be increased.

Referring to FIG. 9 again, the step of providing medical support information (S1000) may further include a step of outputting a list of examination items (S1040). This step may be understood as a step of selecting only the examination items needed for the patient based on the information acquired in the previous steps of performing multi-color analysis and generating a medical report, and showing the examination items to the user (diagnostician). The user (diagnostician) may input several examination items determined to be most urgently required for the patient among the selected examination items so that an examination schedule can be made quickly for the patient.

A method of evaluating an object using color analysis according to the present invention and a system therefor have been described above, and a method of providing a medical support service to a user (diagnostician) by utilizing this evaluation method also has been described above. Meanwhile, the present invention is not limited to the specific embodiments and application examples described above, and various modifications can be made by those skilled in the art without departing from the gist of the present invention claimed in the claims, and these modifications should not be understood as being distinguished from the technical spirit or prospect of the present invention.

### DESCRIPTION OF SYMBOLS

100: Computing device

1. A method of evaluating an object using color analysis based on a biofluorescence image by a computing device including a central processing unit and a memory, the method comprising the steps of:
receiving the biofluorescence image of an arbitrary object;
acquiring RGB values and Lab values of an evaluation area in the biofluorescence image; and
performing color analysis on the basis of the acquired RGB values and Lab values, and displaying a result of the color analysis performed on the object on a specific coordinate system.

2. The method according to claim 1, further comprising, after the step of receiving the biofluorescence image, the step of identifying only the evaluation area from the biofluorescence image.

3. The method according to claim 2, wherein the color analysis includes a process of determining a color cluster to which a plurality of points within the evaluation area belongs by comparing the acquired color information with a previously generated color cluster.

4. The method according to claim 3, wherein the x-axis of the coordinate system represents a ratio of a G value to an R value, and the y-axis represents an L value.

5. The method according to claim 4, wherein the coordinate system includes a plurality of color clusters defined in advance.

6. The method according to claim 1, wherein the step of displaying a result of the color analysis includes a step of displaying points mapped to a plurality of points within the evaluation area on the coordinate system.

7. The method according to claim 6, wherein the step of displaying a result of the color analysis further includes a step of determining to which color clusters the plurality of mapped points belong, and calculating a ratio of the color clusters to which the plurality of points belong.

8. The method according to claim 7, wherein the step of displaying a result of the color analysis further includes a step of determining to which risk group among a high-risk group, a medium-risk group, and a low-risk group the object belongs according to the ratio of the color clusters to which the plurality of points belong.

9. (canceled)

10. A system including a central processing unit and a memory, wherein the central processing unit executes instructions for performing a method of evaluating an object using color analysis stored in the memory, and
the method of evaluating an object using color analysis includes the steps of:
receiving the biofluorescence image of an arbitrary object;
acquiring RGB values and Lab values of an evaluation area in the biofluorescence image; and
performing color analysis on the basis of the acquired RGB values and Lab values, and displaying a result of the color analysis performed on the object on a specific coordinate system.

11. (canceled)

12. (canceled)
